(19) United States
(12) Patent Application Publication
(10) Pub. No.: US 2022/0303662 A1
(43) Pub. Date: Sep. 22, 2022

(54) METHOD FOR SAFE LISTENING AND USER ENGAGEMENT

(71) Applicant: AUDITION TECHNOLOGY, LLC, Pittsburgh, PA (US)

(72) Inventors: Shayan GUPTA, Pittsburgh, PA (US); Hongfu LIU, Pittsburgh, PA (US); Shawn K. KELLY, Pittsburgh, PA (US)

(73) Assignee: AUDITION TECHNOLOGY, LLC, Pittsburgh, PA (US)

(21) Appl. No.: 17/633,949

(22) PCT Filed: Aug. 12, 2020
(86) PCT No.: PCT/US2020/045965

§ 371 Date: Feb. 8, 2022

Related U.S. Application Data
(60) Provisional application No. 62/885,871, filed on Aug. 13, 2019.

(51) Int. Cl. H04R 1/10; H04R 25/00

(52) U.S. Cl. CPC H04R 1/1041; H04R 1/1083; H04R 25/407; H04R 2430/01; H04R 2225/41

(57) ABSTRACT

A system and method for monitoring user sound exposure are disclosed. The system can include receiving audio data generated by an audio source, such as a mobile phone music player, and ambient sound data. Based on this received sound data, the system can determine a cumulative sound exposure for the user, compare the cumulative sound exposure to a threshold, and provide an alert to the user according to the comparison. Based on whether the user adheres to recommended safe listening standards, the system can take additional actions, including providing reports to the user or automatically controlling the volume of the sound that the user is exposed to.

### CROSS REFERENCE TO RELATED APPLICATION

The present application claims priority to U.S. Provisional Patent Application No. 62/885,871, titled “METHOD FOR SAFE LISTENING AND USER ENGAGEMENT,” filed Aug. 13, 2019, which is hereby incorporated by reference herein in its entirety.

### BACKGROUND

Noise-induced hearing loss (NIHL) is a condition caused by exposure to acute or sustained levels of sound that results in damage to the structures in the inner ear, which can lead to temporary or permanent hearing impairment. Although NIHL is not reversible, it is preventable by taking measures such as: (i) increasing awareness of the level and amount of audio to which a person is exposed and the potential for damage, (ii) providing tools to promote safe listening practices, and (iii) empowering users to improve listening habits that can lead to healthy listening behavior. Further, the measures taken to address NIHL must align with evolving demographics and healthcare paradigms, as well as current safe listening standards that are recognized by US federal agencies and international agencies, such as the UN, that set standards for safe listening. Accordingly, there is a need for improvement in the adoption of and user engagement with safe listening practices.

### SUMMARY

In one general embodiment, the systems and methods described herein can be embodied as a combination of a software tool (e.g., an application or app) and associated hardware to promote safe listening as defined by current domestic and international standards. Further, the systems and methods can improve user engagement to promote the safe and effective use of hearing technology, such as personal sound amplification products (PSAPs), hearing aids, and headphones.

In one general embodiment, the present disclosure is directed to a computer-implemented method of monitoring sound exposure for a user using an earpiece and a microphone, the earpiece configured to be communicably connected to a mobile device, the method comprising: receiving, via the microphone, ambient sound data from an environment in which the user is located; receiving audio data generated by an audio source associated with the mobile device and supplied to the earpiece to be emitted thereby; determining a cumulative sound exposure for the user according to the ambient sound data and the audio data; comparing the cumulative sound exposure to a threshold; and providing an alert to the user according to the comparison.

In another general embodiment, the present disclosure is directed to a system for monitoring sound exposure for a user, the system comprising: a microphone configured to receive ambient sound data from an environment in which the user is located; an earpiece configured to communicably connect to a mobile device including an audio source, the mobile device configured to transmit audio data generated by the audio source to the earpiece to be emitted thereby; a processor; and a memory coupled to the processor, the memory storing instructions that, when executed by the processor, cause the system to: receive, via the microphone, the ambient sound data; receive the audio data generated by the audio source; determine a cumulative sound exposure for the user according to the ambient sound data and the audio data; compare the cumulative sound exposure to a threshold; and provide an alert to the user according to the comparison.

### FIGURES

The accompanying drawings, which are incorporated in and form a part of the specification, illustrate the embodiments of the invention and together with the written description serve to explain the principles, characteristics, and features of the invention. In the drawings:

FIG. 1A depicts a block diagram of a first illustrative system for tracking a user's audio exposure in accordance with an embodiment.

FIG. 1B depicts a block diagram of a second illustrative system for tracking a user's audio exposure in accordance with an embodiment.

FIG. 2 depicts a flow diagram of a process for tracking a user's cumulative sound exposure in accordance with an embodiment.

FIG. 3 depicts a flow diagram of a process for tracking a user's cumulative sound exposure in accordance with an embodiment.

FIG. 4 depicts a flow diagram of a process for providing personalized alerts, reports, and other information to the user based on the user's sound exposure profile in accordance with an embodiment.

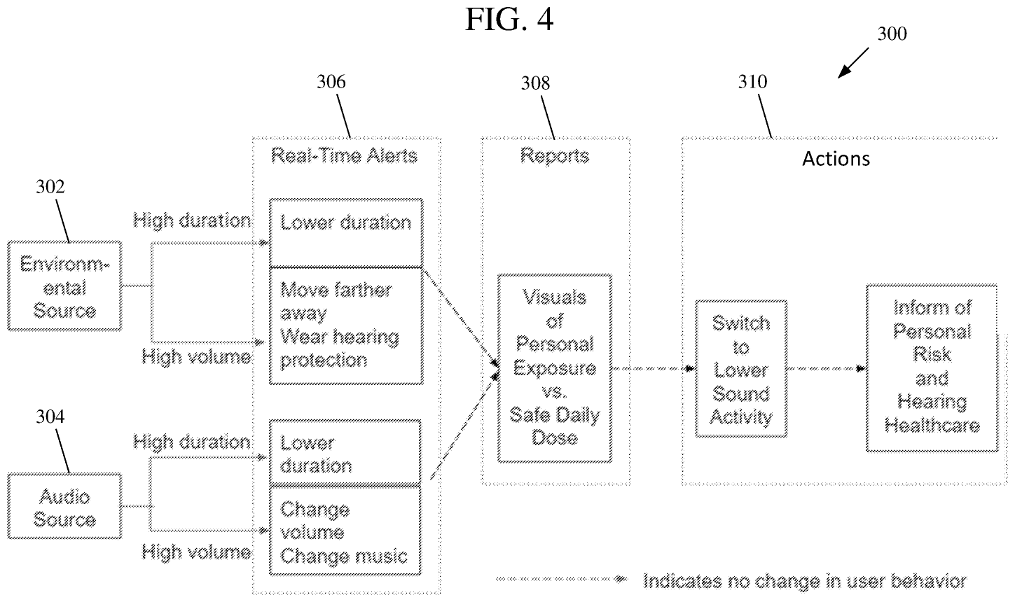

### DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used in this document, “sound” refers to anything audible, whereas “audio” refers to anything audible that has been produced, recorded, or processed by something electronic or digital. Further, as used in this document, “sound data” or “audio data” can include both the data itself and representations that encode or store the data, including digital data, a digital signal, an audio signal, a raw audio or sound recording, and so on.

Described herein are various embodiments of systems and processes for monitoring a user's cumulative exposure to both ambient sound and sound generated by an audio source. The system can include a mobile device, such as a smartphone, and an earpiece, such as a hearing aid (e.g., a behind-the-ear (BTE), mini-BTE, or over-the-counter hearing aid), a PSAP, or headphones. In a general embodiment, the system functions by receiving or obtaining ambient sound data using a microphone and receiving or obtaining sound data generated by an audio source (e.g., a mobile device) that is to be supplied to the earpiece to be emitted thereby. By monitoring both the ambient sound and the sound data generated by the audio source, the system can track the cumulative amount and/or level of audio that the user is exposed to over a particular time period and provide recommendations and/or alerts to the user accordingly. The microphone can be associated with or integrated into the mobile device or the earpiece. The audio source sound can include, for example, music generated by an audio player.

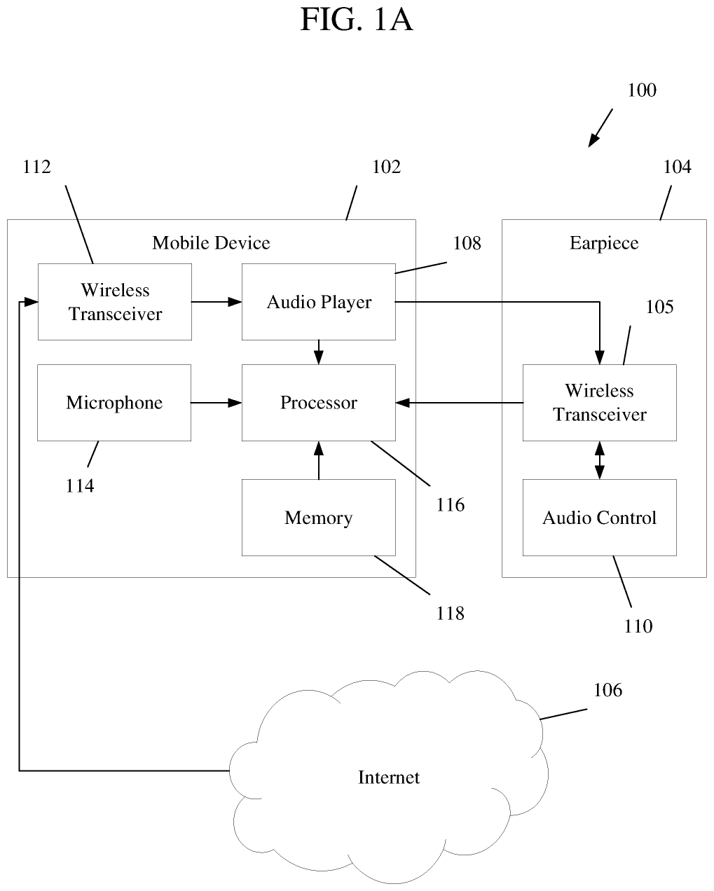

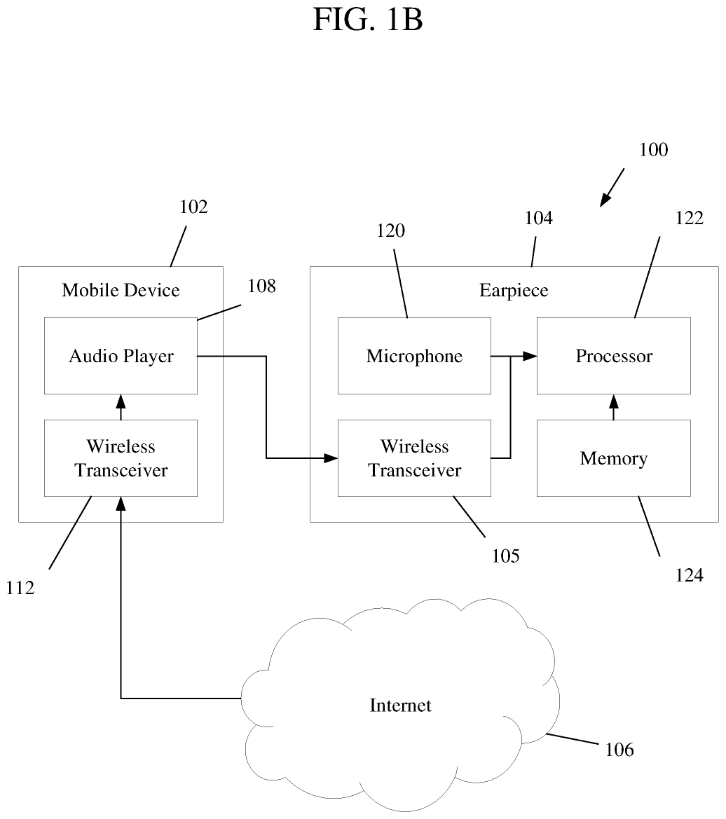

Referring now to FIGS. 1A and 1B, a user audio monitoring system 100 can include a mobile device 102 and an earpiece 104. The earpiece 104 can include a wireless transceiver 105 configured to communicably connect to the mobile device 102 using a variety of different connection types and/or communication protocols (e.g., Bluetooth). The earpiece 104 can be configured to convert electronic signals into sound pressure waves that are intended to be emitted into a user's auditory canal. In an embodiment, the earpiece 104 can be configured to receive audio signals or data from the mobile device 102, convert the audio signals or data into audio to be provided to the user, and then emit the generated audio. Further, the earpiece 104 can include various software and/or hardware systems that are configured to amplify and/or modulate audio signals received from the mobile device 102. In one embodiment, the mobile device 102 can be configured to transmit sound data directly to the earpiece 104, such that the transmitted sound data is modified only by the amplification and modulation systems of the earpiece 104 before being presented to the user.

The mobile device 102 can be configured to store and execute software applications (i.e., apps) that can generate audio that is to be presented to a user. These apps can include an audio player 108 that is configured to download or stream music, such as Spotify, iTunes, or Google Play Music. The mobile device 102 can include a wireless transceiver 112 that allows the audio player 108 to download or stream music or other audio (e.g., podcasts) via the Internet 106 (or another communication network). However, the mobile device 102 can store and execute a variety of different sound data-generating apps that are not limited solely to music downloading or streaming apps.

As will be described in greater detail below, the mobile device 102 and/or earpiece 104 can be configured to individually or collectively execute a process to monitor the cumulative amount or level of audio to which the wearer of the earpiece 104 is exposed. The audio monitoring system 100 can use a microphone 114 (which can be associated with either the mobile device 102 or the earpiece 104) to sample ambient sound in the user's environment. The audio monitoring system 100 can further sample the audio generated by the mobile device 102 (e.g., by an audio player 108) that is to be provided to the earpiece 104 for the user. The audio monitoring system 100 can use various transfer functions to calculate the user's cumulative sound exposure over a particular time period. In one embodiment, the audio monitoring system 100 can calculate the user's daily audio exposure. The audio monitoring system 100 can compare the cumulative sound exposure to one or more audio exposure thresholds. Illustrative audio exposure thresholds include the revised criteria for occupational noise exposure issued by the National Institute for Occupational Safety and Health (NIOSH) of the United States, the global standard for safe listening devices and systems issued by the World Health Organization-International Telecommunications Union (WHO-ITU), and other US standards promulgated by the Centers for Disease Control and Prevention (CDC), Occupational Safety and Health Administration (OSHA), National Institute for Deafness and Communication Disorders (NIDCD), Environmental Protection Agency (EPA), Department of Defense Hearing Center of Excellence (DoD-HCE), Army Research Lab (ARL), and Army Public Health Center (APHC). This comparison between the user's cumulative sound exposure and the one or more audio exposure thresholds can be used to provide the user with individualized alerts, recommendations, and/or other feedback. In various embodiments, the user feedback may include alerts provided by the mobile device 102 (e.g., using push notifications), haptic feedback from the mobile device 102 and/or earpiece 104, and so on.

The user audio monitoring system 100 described above can take a variety of different forms. FIGS. 1A and 1B show different illustrative embodiments for this user audio monitoring system 100.

In the embodiment of the audio monitoring system 100 shown in FIG. 1A, the mobile device 102 receives or samples ambient sound using a microphone 114 associated with the mobile device. Further, the mobile device 102 receives or samples audio generated by an audio source, which can include the mobile device itself or another audio source (e.g., an audio player 108 executed by the mobile device). In one embodiment, the audio generated by the audio source is provided to the mobile device 102 after it has been modulated by an audio control system 110 of the earpiece 104.

The microphone 114 can include an internal or external microphone of the mobile device 102. The microphone 114 can be positioned or otherwise configured to receive ambient sound from the environment in which the mobile device 102 is located. Further, in the embodiment depicted in FIG. 1A, the earpiece 104 can be communicably coupled to the mobile device 102 (e.g., via a wireless transceiver 105) such that music or other sound data generated by the audio player 108 is received by the earpiece 104 from the mobile device. The audio control system 110 can be configured to control, for example, a volume level of the audio associated with the audio data received from the mobile device 102. The earpiece 104 can be configured to emit audio to the user based on the received sound data, either as received from the mobile device 102 or as modified by the audio control system 110. In one embodiment, the post-processed audio data generated by the audio control system 110 can be transmitted back to the mobile device 102 (e.g., via the wireless transceiver 105) for analysis thereby.

The embodiment shown in FIG. 1A can be beneficial because it allows the audio monitoring system 100 to leverage the ubiquity and convenience of mobile devices 102. Further, in embodiments where the processes described below are embodied as software apps stored on and executed by the mobile device 102, software updates to the apps can be easily pushed to users' mobile devices through existing app update systems.

The embodiment of the audio monitoring system 100 shown in FIG. 1B differs from the embodiment shown in FIG. 1A in that the earpiece 104 contains hardware components in addition to or in lieu of the hardware components shown in FIG. 1A. Accordingly, all or a portion of the process of monitoring a user's audio exposure can be performed onboard the earpiece 104. In the embodiment shown in FIG. 1B, the earpiece 104 can include a microphone 120 that is positioned or otherwise configured to receive ambient sound from the environment in which the earpiece 104 is located. The microphone 120 can be communicably coupled to a processor 122 such that the processor can receive the sampled audio and/or sound data from the microphone 120. Further, the earpiece 104 can be communicably coupled to the mobile device 102 (e.g., via a wireless transceiver 105) such that music or other sound data generated by the audio player 108 is received by the earpiece 104 from the mobile device. The earpiece 104 can be configured to emit audio to the user based on the received sound data.

The embodiment shown in FIG. 1B can be beneficial because it allows all or a substantial amount of the audio processing and monitoring to be performed on the earpiece 104 itself. This removes the need to rely upon the mobile device 102, which may be undesirable for some users. Further, the embodiment of the audio monitoring system 100 can make use of edge computing or distributed computing techniques to improve the data processing efficiency.

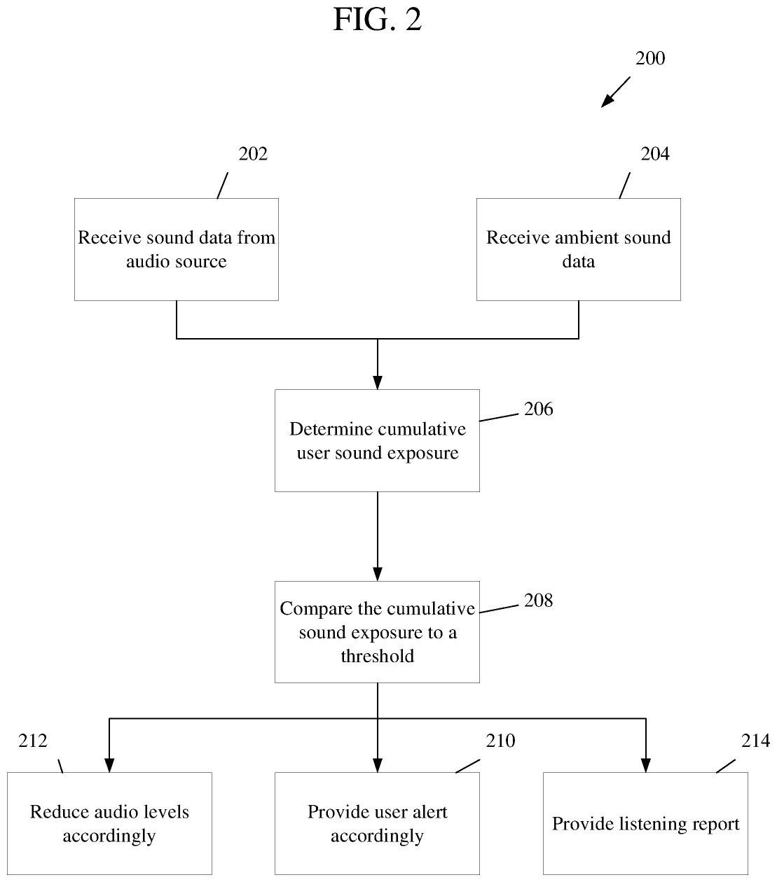

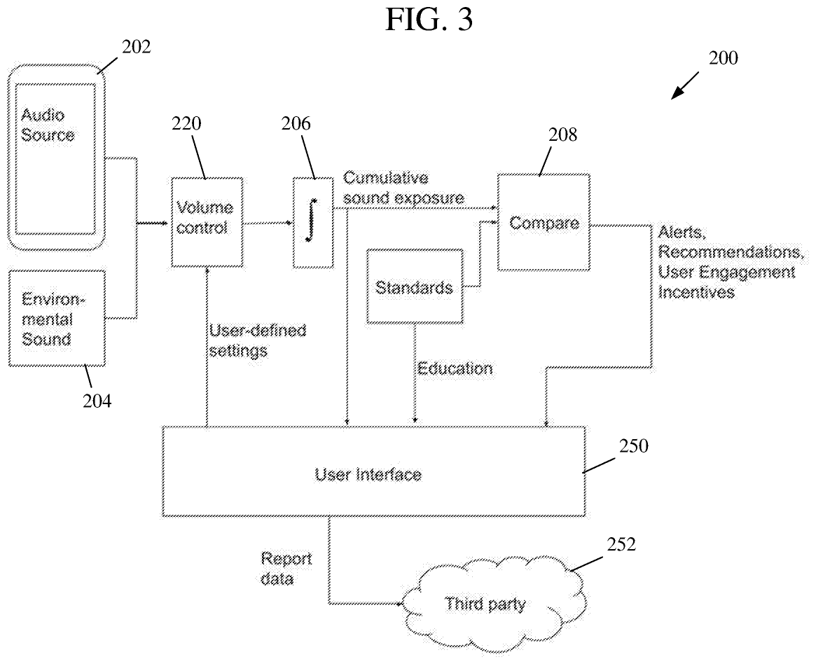

The various embodiments of audio monitoring systems 100 described above can be used to monitor a user's cumulative exposure to both ambient sound and audio generated by the mobile device 102. FIG. 2 depicts a flow diagram of an illustrative computer-implemented process 200 for monitoring the cumulative sound exposure to which a user is exposed. In the following description of the process 200, reference should also be made to FIG. 3. The process 200 can be executed by a computer (e.g., a mobile computing device). Further, the process 200 can be embodied as software, hardware, firmware, or combinations thereof. In one embodiment, the process 200 can be embodied as instructions stored in a memory that, when executed by a processor coupled to the memory, cause a computer to perform the one or more steps of the process 200. In one embodiment, the process 200 can be embodied as a software application (e.g., a smartphone app) executed by a processor 116 of a mobile device 102, such as is shown in FIG. 1A. In another embodiment, the process 200 can be executed by a processor 122 of an earpiece 104, such as is shown in FIG. 1B. In yet another embodiment, the mobile device 102 and the earpiece 104 can be components of a distributed computing system and, accordingly, the process 200 can be executed by the combination of the devices. In the following description of the process 200, the “device” executing the process 200 can refer to a computer system, a mobile computing device, a mobile device 102, an earpiece 104, and/or the like.

As noted above, in one embodiment, the process 200 can be embodied as a software app. The software app can be used as a companion for an earpiece 104 and can be configured to prompt users to make informed decisions about personal listening behaviors based on personalized listening trends. The app can periodically (e.g., throughout a day) monitor the amounts or levels of ambient sound and audio source sound that the user has been exposed to in order to estimate the user's personalized sound exposure. In an embodiment, the software app can present alerts and notifications to the user to indicate how the user's personal listening behavior compares to sound doses prescribed by safe listening standards. In an embodiment, the software app can also provide personalized user recommendations, such as a recommendation that the user limit or counteract unsafe noise exposure based on the user's daily lifestyle as determined from the received ambient sound data and audio source sound data.

Accordingly, a device executing the process 200 can receive 202 sound data that is generated by an audio source (e.g., the mobile device 102 or an audio player 108 executed thereby) and that is transmitted or otherwise provided by the audio source to the earpiece 104 to be emitted to the user. As described above, the earpiece 104 can, in some embodiments, include an onboard audio control 110 that is configured to process or modify the audio data that is received from the mobile device 102 (e.g., increase or decrease the volume). Accordingly, in one embodiment, the received 202 sound data can include sound data that has been post-processed by the earpiece 104 (e.g., the audio data that has been processed by the audio control 110 of the earpiece 104). This embodiment can be beneficial because it allows the audio control system 100 to determine the actual sound that the user is being exposed to.

In addition, the device can receive 204 ambient or environmental audio. The device can receive 204 the ambient sound via a microphone, such as a microphone 114 associated with the mobile device 102 or a microphone 120 associated with the earpiece 104. In various embodiments, the received 202, 204 audio source sound data and ambient sound data can be in the form of digital data, an audio signal, raw audio, and other formats. In various embodiments, the sound data can include, for example and without limitation, a volume level, amplitude/frequency data, a music genre, and the like.

In various embodiments, the process 200 can include controlling 220 a volume of the audio source sound data and/or ambient sound data or making other modifications to the received 202, 204 sound data. In one embodiment, the volume control setting may be controlled 220 through a user interface 250, as shown in FIG. 3. The user interface may include a graphical user interface or other interface types. The user interface 250 may be provided by or through, for example, a smartphone app. As described further below, the audio source sound data and/or ambient sound data may be controlled 220 in response to a user not taking appropriate corrective actions as dictated by notifications or reports provided by the audio monitoring system 100.

Accordingly, the device can determine 206 a cumulative sound exposure for the user based on the ambient sound data and the mobile device sound data. The cumulative sound exposure can be based on the volume level and/or sound pressure exposure that the user has been exposed to, as determined from the ambient sound data and the mobile device sound data. The volume level can be expressed in decibels (dB), for example. In one embodiment, the device can calculate a cumulative sound exposure metric for the user. In one embodiment, the device can calculate an A-weighted decibel value (dBA) or another such metric configured to account for relative or perceived loudness of sound. In one embodiment, the calculated cumulative user sound exposure may be provided to the user through the user interface 250.

Accordingly, the device can compare 208 the determined cumulative user sound exposure to one or more safe listening thresholds. The thresholds can be based on, for example, various domestic and international safe listening standards, some of which are described above. Further, the thresholds can be defined in terms of individual variables (e.g., a particular sound level) or combinations of variables (e.g., a particular sound level over a particular period of time). In one embodiment, the thresholds can be set or adjusted by user preferences. For example, a user may establish a user profile including safe listening settings. In one embodiment, the user profile may be established or modified using the user interface 250, as shown in FIG. 3. The user interface 250 may be provided by or through, for example, a smartphone app. Based on the results of the comparison between the cumulative user sound exposure and the one or more thresholds, the device executing the process 200 can take a variety of different actions or can take no action at all.

In one embodiment, the device executing the process 200 may provide 210 an alert to the user if, for example, the cumulative user sound exposure is outside of one or more of the relevant thresholds. The user alert may be embodied as a push notification provided 210 via a software app, haptic feedback, audible feedback, and so on. The type of alerts provided 210 by the device may be customized according to the user's preferences and controlled through the user interface 250, for example. In one embodiment, the user alert may indicate a maximum amount of time that the user should continue being subjected to the current cumulative sound exposure level.

In one embodiment, the device executing the process 200 may reduce 212 the audio level of the audio source if, for example, the cumulative user sound exposure is outside of one or more of the relevant thresholds. For example, the device may automatically reduce 212 the audio level associated with the audio player 108 or the mobile device 102 so that the sound generated by the mobile device 102 is within the one or more safe listening thresholds. In one embodiment, an alert or notification (e.g., a push notification) may be provided to the user prior to the audio level of the audio source being reduced 212.

In one embodiment, the device executing the process 200 may provide 214 a report to the user and/or a third party. In one embodiment, a report may be provided 214 to the user at a regular interval (e.g., daily or weekly). In an alternate embodiment, a report may be provided 214 within a time period after the cumulative user sound exposure being outside of one or more of the relevant thresholds. The report may include, for example and without limitation, data associated with the sound levels to which the user has been exposed; recommendations for the user to take actions to address sound exposure volume, duration, distance, and/or the like; one or more alternative listening options; and/or a recommendation to wear or use hearing protection. In an embodiment, a recommendation may be based on an analysis of the user's sound exposure behaviors, including the user's listening behaviors with respect to sound generated by an audio source (e.g., the user's average music listening volume) or the user's pattern of environmental sound exposure (e.g., whether the user is regularly exposed to unsafe levels of sound, such as jet engines, construction noises, and so on). In some embodiments, a recommendation may include, for example and without limitation, to lower their phone volume, to listen to music of a different genre, to use hearing protection, and/or to shorten exposure duration by suggesting breaks and alternative sound exposure options (e.g., as guided by daily activities). In some embodiments, illustrative recommendations may further include education and preventative measures to improve the user's hearing wellness. For example, the education information may include information on NIHL from medical, federal, military and regulatory sources. The education information may disclose, for example and without limitation, causes of hearing loss, individuals that could be at risk, and current standards that regulate noise exposure. The device and/or software application may enable the user to access educational materials related to various hearing healthcare topics relevant to the user's lifestyle, provide access options to check the user's hearing (e.g., connect or link to the hearWHO app), provide one or more reminders to visit a hearing healthcare professional, provide one or more recommendations for selecting hearing protection based on the user's needs and preferences, and provide information on other recommended practices aimed at preventing hearing loss. Further, recommendations for personal improvements may include, for example and without limitation, qualitative recommendations (e.g., personal summaries of changes/improvements in listening practices or a user's changes in music practices over time) and quantitative recommendations (e.g., personal sound exposure metrics indicating whether a user's sound exposures are aligned with safe listening standards, healthy listening scores, or residual hearing metrics).

As noted above, reports can be provided 214 directly to the user (e.g., via a push notification). In other embodiments, the reports may be provided 214 (e.g., as authorized by the user) to a third party 252, as shown in FIG. 3. The third party receiving the reports may include, for example and without limitation, a family member, a medical practitioner, a school, or an organization maintaining occupational requirements for the user. The report provided 214 to the third party may include information customized by the user, such as personal sound exposure metrics and recommended changes in user listening habits.

In one embodiment, the device and/or the software executed by the device may include privacy and security measures to safeguard the user's personal information, such as limiting data collection to that required specifically for the execution of the process 200 described above and implementing relevant data protection regulations as required by the Health Insurance Portability and Accountability Act (HIPAA), the General Data Protection Regulation (GDPR), and other domestic or international regulations.

As noted above, the user can control certain settings or parameters, such as the user alert thresholds, user alert types, and the provided reports. These and other settings can be saved or otherwise associated with a personal user profile for each user. One of the main goals of the systems and methods described herein is to encourage users to actively engage in the management of their own hearing health by personalizing the recommendations and information that are provided to the user based on each user's user profile and personalized sound exposure profile. The three main approaches for personalizing each user's experience include (i) allowing users to actively control and customize the monitoring by the systems described herein, (ii) facilitating each user's awareness of their personal sound exposure, and (iii) providing personalized feedback to the user. For example, users can manage the sound monitoring by the system by allowing users to select the intervals at which noise exposure is sampled and logged into the cumulative exposure assessment. This flexibility improves the accuracy of the sound monitoring system by providing recommendations to the user to change the sampling rate according to the user's sound exposure profile. Further, this flexibility allows users to select one or more times during the day at which to sample the user's sound exposure based on daily habits. The user also has the flexibility of choosing an interval for alerts and recommendations. This allows the user to select one or more times when the information will be useful and likely to be acted upon. Further, personalized real-time exposure monitoring overcomes situations in which a user cannot perceive noise exposures that have the potential to impair hearing. For example, short-term loud volume levels (e.g., impact sounds) or long-term exposure to seemingly tolerable sound levels may not cause discomfort that would otherwise alert the user to unsafe exposure.

Correspondingly, the systems and methods described herein can estimate each individual user's personal sound exposure profile, through defined standards, which can be used to assess the risk of such exposures. The systems can be configured to calculate or estimate the user's sound exposure in real time and, correspondingly, provide real-time feedback to alert the user to potentially damaging exposure levels so that the user can take immediate corrective action. In addition to monitoring for acute sound exposure events, the systems can also track the cumulative sound exposure of the user over particular period of times, which can similarly be used to provide feedback to alert the user to potential risks based on the cumulative exposure duration. Personalized feedback can be derived from trends in the user's real-time sound pressure level and cumulative sound pressure exposure. The user's real-time exposure and recorded listening behavior trends are compared to the user's desired sound exposure, as per one or more relevant standards. The generated feedback addresses outliers in the user's sound exposure with suggested recommendations to address the unsafe exposures.

In some embodiments, feedback mechanisms may be evaluated to determine whether a particular type of feedback resulted in a change in listening behavior as a means of assessing the usefulness of that type of feedback to the user. In some embodiments, if a previous type of feedback did not produce the desired change in listening behavior, a new type of feedback offering different solutions may be presented to the user.

In an embodiment where the process 200 described above is embodied as a smartphone app, the app may include a visual user interface 250 that displays a particular sequence of information after the user logs in. In particular, the app may provide, for example and without limitation, a user profile selection, a personal sound exposure report, any alerts with corresponding recommendations, and personalized education materials based on the user's profile and sound exposure report. In some embodiments, the app may further include or provide appropriate measures for data privacy, any necessary permissions for data sharing, and cybersecurity recommendations.

In some embodiments, the user profile page may include various settings that can be selected or controlled by the user, such as a decibel meter, a listening profile, a hearing history, and listening essentials. The decibel meter may include, for example and without limitation, displays identifying information pertaining to real-time, daily, and/or weekly sound exposures. Real-time alerts can be displayed without the user needing to open the app and can include additional information, such as a timer for the maximum duration permissible for hearing impairment could result. The alerts can include, for example and without limitation, push notifications, pop-up messages, and audible indicators, such as beeps. The listening profile may allow the user to designate one or more personal features, such as sources of sound exposure, occupation, sports played, workout times, when and what types of entertainment the user is participating in, any home projects being performed by the user, timing that the user wants the app to monitor sound exposure for, frequency of alerts, alert types, and frequency of cues for safe listening. In one embodiment, personal sound exposure reports can use various graphical displays of real-time (e.g., captured in 1 second intervals), daily, and/or weekly sound exposure reports; any alerts provided by the app; any detected user response to the alerts or recommendations; and user hearing scores, such as hearWHO listening scores.

As described above, different alerts, reports, and other information provided to the user can be triggered based on each user's personalized sound exposure profile. For example, FIG. 5 shows a flow diagram of one illustrative process 300 for providing personalized alerts, reports, and other information to the user based on the user's sound exposure profile. As described above, the audio monitoring system 100 is configured to monitor sounds from both environmental sources 302 and audio sources 304 (e.g., an audio player 108, such as a music streaming service, on a mobile device 102). The overall input to the audio monitoring system 100 is the sound/audio data from environmental sources 302 and audio sources 304. The overall outputs of the audio monitoring system 100 can include visual, haptic, and other feedback that is intended to alter the user's safe listening behaviors, provide reports and other information to the user so that the user can identify whether one or more listening behaviors create risks and which risks are created, and provide choices to the user for improving their hearing health. The different sources of the audio input and the detected change or lack of change in user behavior may trigger different output feedback.

In one embodiment, the first type of information provided to the user can include real-time alerts 306 (e.g., push notifications). The type of real-time alert 306 provided to the user can vary based upon a variety of different parameters associated with the audio source data and the ambient sound data, such as the duration and volume of the sampled sound. For example, if the user is exposed to a sustained, high duration sound from an environmental source 302 that triggers relevant safe listening thresholds, the real-time alert 306 can include a recommendation for the user to reduce the duration of exposure to the sound. If the user is being exposed to a high volume sound from an environmental source 302 that triggers the relevant safe listening thresholds, the real-time alert 306 can include a recommendation for the user to move away from the environmental source 302 and/or wear hearing protection. If the user is being exposed to a sustained, high duration sound from an audio source 304 that triggers the relevant safe listening thresholds, then the real-time alert 306 can include a recommendation for the user to reduce the duration of exposure to the sound (e.g., by listening to the music from their audio player 108 for a shorter period of time). If the user is being exposed to a high volume sound from an audio source 304 that triggers the relevant safe listening thresholds, then the real-time alert 306 can include a recommendation for the user to decrease the volume of the audio source 304 and/or change the music to which they are listening (e.g., switch the genre of music to which they are listening).

If the sound data from the environmental source 302 and/or the audio source 304 continues to exceed the relevant safe listening thresholds despite the provided real-time alerts 306, the audio monitoring system 100 can provide reports 308 to the user. The reports 308 can represent a graduated response to further encourage the user to engage in safe listening behaviors. The reports 308 can include, for example and without limitation, push notifications, emails, and information relayed using the user interface 250. For example, if the sound data from the environmental source 302 and/or the audio source 304 continues to exceed the relevant safe listening thresholds after the real-time alerts 306 have been provided, then the report 308 can include a visualization of the user's sound exposure relative to safe listening standards for a particular time period (e.g., daily). This visualization may, for example, show when and by how much the user's sound exposure is exceeding safe listening standards to further encourage the user to make behavioral changes to promote their hearing health.

If the sound data from the environmental source 302 and/or the audio source 304 continues to exceed the relevant safe listening thresholds despite the provided real-time alerts 306 and the provided reports 308, the audio monitoring system 100 can take additional actions 310. For example, the audio monitoring system 100 can decrease the volume of the audio source 304 or otherwise switch the audio source 304 to a lower sound activity. In one embodiment, the audio monitoring system 100 can automatically decrease the volume of the audio source 304. In another embodiment, the audio monitoring system 100 can provide the user with the option to decrease the volume of the audio source 304, thereby giving the user a choice. In an embodiment, the audio monitoring system 100 can inform the user (e.g., via an email, push notification, or information relayed using the user interface 250) of the personal risk to their hearing health (e.g., the risk that they could develop NIHL) or refer the user to hearing healthcare resources. In one embodiment, the audio monitoring system 100 can provide the user information of the personal risk to their hearing health and/or refer the user to hearing healthcare resources in the event that the user elected not to decrease the volume from the audio source 304 or otherwise declined to change their listening behaviors.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A computer-implemented method of monitoring sound exposure for a user using an earpiece and a microphone, the earpiece configured to be communicably connected to a mobile device, the method comprising:
receiving, via the microphone, ambient sound data from an environment in which the user is located;
receiving audio data generated by an audio source associated with the mobile device and supplied to the earpiece to be emitted thereby;
determining a cumulative sound exposure for the user according to the ambient sound data and the audio data;
comparing the cumulative sound exposure to a threshold; and
providing an alert to the user according to the comparison.

2. The computer-implemented method of claim 1, further comprising:
if the cumulative sound exposure continues to exceed the threshold after the user alert was provided, providing a report to the user, the report visualizing the comparison between the cumulative sound exposure to the threshold.

3. The computer-implemented method of claim 2, further comprising:
decreasing a volume of the audio source according to the comparison.

4. The computer-implemented method of claim 2, further comprising:
providing healthcare information to the user according to the comparison.

5. The computer-implemented method of claim 1, wherein the mobile device comprises the microphone.

6. The computer-implemented method of claim 1, wherein the earpiece comprises the microphone.

7. The computer-implemented method of claim 1, wherein the earpiece is selected from the group consisting of a hearing aid and a personal sound amplification product.

8. The computer-implemented method of claim 1, wherein the alert comprises at least one of a push notification, an email, a text message, or information displayed by a user interface of a software application executed by the mobile device.

9. The computer-implemented method of claim 1, wherein the audio source comprises a music streaming application.

10. The computer-implemented method of claim 1, wherein the threshold corresponds to at least one of a volume level of the cumulative sound exposure or a duration of the cumulative sound exposure.

11. A system for monitoring sound exposure for a user, the system comprising:
a microphone configured to receive ambient sound data from an environment in which the user is located;
an earpiece configured to communicably connect to a mobile device including an audio source, the mobile device configured to transmit audio data generated by the audio source to the earpiece to be emitted thereby;
a processor; and
a memory coupled to the processor, the memory storing instructions that, when executed by the processor, cause the system to:
receive, via the microphone, the ambient sound data;
receive the audio data generated by the audio source;
determine a cumulative sound exposure for the user according to the ambient sound data and the audio data;
compare the cumulative sound exposure to a threshold; and
provide an alert to the user according to the comparison.

12. The system of claim 11, the memory further storing instructions that, when executed by the processor, cause the system to:
if the cumulative sound exposure continues to exceed the threshold after the user alert was provided, provide a report to the user, the report visualizing the comparison between the cumulative sound exposure to the threshold.

13. The system of claim 12, the memory further storing instructions that, when executed by the processor, cause the system to:
decrease a volume of the audio source according to the comparison.

14. The system of claim 12, the memory further storing instructions that, when executed by the processor, cause the system to:
provide healthcare information to the user according to the comparison.

15. The system of claim 11, wherein the mobile device comprises the microphone.

16. The system of claim 11, wherein the earpiece comprises the microphone.

17. The system of claim 11, wherein the earpiece is selected from the group consisting of a hearing aid and a personal sound amplification product.

18. The system of claim 11, wherein the alert comprises at least one of a push notification, an email, a text message, or information displayed by a user interface of a software application executed by the mobile device.

19. The system of claim 11, wherein the audio source comprises a music streaming application.

20. The system of claim 11, wherein the threshold corresponds to at least one of a volume level of the cumulative sound exposure or a duration of the cumulative sound exposure.

21. The system of claim 11, wherein the mobile device comprises the processor and the memory.

22. The system of claim 11, wherein the earpiece comprises the processor and the memory.
